(19) United States
(12) Patent Application Publication
(10) Pub. No.: US 2022/0378840 A1
(43) Pub. Date: Dec. 1, 2022

(54) COMPOSITIONS, KITS AND METHODS FOR STORAGE OF BLOOD PRODUCTS AND METHODS OF USE THEREOF

(71) Applicant: Rutgers, The State University of New Jersey, New Brunswick, NJ (US)

(72) Inventors: Wise Young, New Brunswick, NJ (US); Dongming Sun, Princeton Junction, NJ (US); Iman Tadmori, Berkely Heights, NJ (US)

(73) Assignee: Rutgers, The State University of New Jersey, New Brunswick, NJ (US)

(21) Appl. No.: 17/755,955

(22) PCT Filed: Nov. 25, 2020
(86) PCT No.: PCT/US2020/070819

§ 371 Date: May 12, 2022

Related U.S. Application Data
(60) Provisional application No. 62/942,060, filed on Nov. 29, 2019.

(51) Int. Cl. A61K 35/18; A61K 35/51; A61K 38/01

(52) U.S. Cl. CPC A61K 35/18; A61K 35/51; A61K 38/017

(57) ABSTRACT

Compositions, containers and kits for low temperature storage of a specified quantity of packed red blood cells (RBCs) include L-camitine, HES and blood plasma proteins. Methods of storing a packed RBC blood product, transfusing a packed RBC blood product into a subject, and treating anemia, ischemia, hypoxia, a hemoglobin disorder, or a hematopoietic disorder in a subject (e.g., a human) include the compositions, containers, kits and blood products.

### CROSS REFERENCE TO RELATED APPLICATION

This application claims priority to U.S. Provisional Applications Nos. 62/942,060 filed on Nov. 29, 2019, which is incorporated by reference herein in its entirety.

### FIELD OF THE INVENTION

The invention relates generally to the field of medicine. In particular, the invention relates to compositions, containers, kits and methods for storing blood products and methods of use thereof.

### BACKGROUND

Red blood cells (RBCs) have been used to treat ischemia due to hemorrhage or hemorrhagic shock. Umbilical cord blood (UCB) RBCs are a particularly good treatment for infants with anemia, hypoxia or ischemia, or infants with hematopoietic disorders such as thalassemia and sickle cell anemia. RBCs are usually stored for 2 weeks at 4° C. after collection and then discarded or frozen in high concentrations of organic solvents, i.e. 20-50% glycerol or 10% dimethylsulfoxide (DMSO). Due to toxicity of these solvents, the RBCs must be frozen shortly after adding glycerol or DMSO and washed soon after thawing. Cold RBCs lose potassium and deplete their ATP stores restoring their potassium gradient when thawed. Improved methods of cryopreserving blood products such as RBCs that avoid ice crystal formation and that do not require a washing step are needed.

### SUMMARY

This invention addresses the problems associated with known blood product storage methods by combining blood cells (e.g., RBCs) with L-carnitine (a natural amino acid, zwitterion, and antioxidant), hydroxyethyl starch (HES), and blood plasma proteins (e.g., human serum albumin (HSA)) to preserve the blood cells (e.g., RBCs) at 4° C. and freezing temperatures. L-carnitine and HES, both non-toxic natural substances, are included as cryopreservants. L-carnitine is a common nutrient and dietary supplement that is made in the body and has very low toxicity. HES has long been used to treat patients with hemorrhagic shock and as a volume replacer in children. The L-carnitine and HES enter blood cells (e.g. RBCs) to prevent intracellular ice crystal formation. The blood plasma proteins (e.g., albumin, HSA) increase extracellular osmolarity to draw water out of the cells. In a typical embodiment, the blood cells are RBCs and packing RBCs reduces extracellular space and K loss. In such embodiments, shortly after collection, RBCs are placed in the sterile freezing containers (sterile freeze-resistant containers) described herein that contain freeze-dried L-carnitine, HES, and HSA, stored 2 weeks in case their use is required, and frozen if not needed. The methods of storing a packed RBC blood product as described herein increase RBC survival during cryopreservation and reduce washing of the RBCs after thawing. The methods provide storage and freezing of blood cells (e.g., RBCs) from UCB in the absence of toxins and are suitable for treating diseases such as ischemia in a subject, and anemia in newborn infant subjects. Blood cells (e.g., RBCs) stored according to the methods described herein are expected to be more active metabolically, more durable, and longer-lived after transplantation into a subject in need thereof than blood cells (e.g., RBCs) frozen in glycerol solutions. In the methods of transfusing a packed RBC blood product into a subject described herein, UCB RBCs deliver more oxygen than adult cells to the target area, e.g., ischemic or hypoxic brain or organs.

Accordingly, described herein is a kit for low temperature storage of a specified quantity of packed RBCs. The kit includes a freeze-resistant sterile container that includes L-carnitine, hydroxyethyl starch (HES), and blood plasma proteins, wherein the L-carnitine and HES are present in quantities effective to provide cryoprotection for the specified quantity of packed RBCs and the blood plasma proteins are present in a quantity effective to reduce RBC edema for the specified quantity of packed RBCs. The kit also includes packaging and instructions for use. The kit can further include packed RBCs, e.g., UCB RBCs. In such kits, the packed RBCs can be essentially free of white blood cells. In some embodiments of the kit, the blood plasma proteins are HSA. In some embodiments of the kit, the L-carnitine, HES and blood plasma proteins are freeze-dried. In some embodiments, the HES has a molecular weight between about 130 and about 200 kDa (e.g., about 130 to about 135 kDa) and between about 35% and about 50% (e.g., about 40% to about 45%) of the starch glucose units have been modified with hydroxyethyl groups. In the kit, the L-carnitine, HES and blood plasma proteins can be combined in a composition, or they can be added to the sterile container individually.

Also described herein is a method of storing a packed RBC blood product, the method including the steps of:
i. mixing the blood product with L-carnitine, HES and blood plasma proteins in a freeze-resistant sterile container as described herein;
ii. incubating the blood product preparation in the sterile container for a period of time effective to provide cryoprotection to the blood product and reduce RBC edema; and
iii. freezing the sterile container and the blood product at a temperature between about −20° C. and about −180° C.

In this method, the phrase “incubating the blood product preparation in the sterile container for a period of time effective to provide cryoprotection to the blood product and reduce RBC edema” of step ii. means incubating the cells under conditions that allow the cells to equilibrate such that the cells take up the L-carnitine, HES and blood plasma proteins and the cells become cryoprotected. Such conditions are well known in the art. Such conditions can include incubating the blood product preparation for approximately two weeks (e.g., 12 days, 13 days, 14 days, 15 days). Also in this method, step iii. of freezing the blood product can include a controlled rate of freezing of about 1° C. per minute. Also in the method, the RBCs can include UCB cells and/or the blood plasma proteins can be HSA. In some embodiments of the method, the L-carnitine, HES and blood plasma proteins are freeze-dried. In some embodiments, the HES has a molecular weight between about 130 and about 200 kDa (e.g., about 130 to about 135 kDa) and between about 35% and about 50% (e.g., about 40% to about 45%) of the starch glucose units have been modified with hydroxyethyl groups.

Further described herein is a method of transfusing a packed RBC blood product into a subject. The method includes thawing, washing, and reconstituting the blood product stored according to the methods described herein, and infusing the reconstituted blood product into the subject, wherein said blood product is ABO-matched to the subject. In the method, the RBCs can include UCB cells, and the blood plasma proteins can be HAS (e.g., consist essentially of HSA). In some embodiments, the L-carnitine, HES and blood plasma proteins are freeze-dried, and/or the HES has a molecular weight between about 130 and about 200 kDa (e.g., about 130 to about 135 kDa) and between about 35% and about 50% (e.g., about 40% to about 45%) of the starch glucose units have been modified with hydroxyethyl groups.

Still further described herein is a method of treating a subject suffering from anemia, ischemia, hypoxia, a hemoglobin disorder, or a hematopoietic disorder. The method includes thawing, washing and reconstituting a packed RBC blood product stored according to the methods described herein, and infusing the reconstituted blood product into the subject, wherein said blood product is ABO-matched to the subject. In some embodiments of the method, the blood product includes cells from the subject. In some embodiments of the method, the RBCs include UCB cells, and/or the blood plasma proteins are HAS, and/or the L-carnitine, HES and blood plasma proteins are freeze-dried. In some embodiments, the HES has a molecular weight between about 130 and about 200 kDa (e.g., about 130 to about 135 kDa) and between about 35% and about 50% (e.g., about 40% to about 45%) of the starch glucose units have been modified with hydroxyethyl groups. In the method, the subject (e.g., a human infant) can be suffering from one or more of the following disorders: ischemia, hypoxia, anemia, thalassemia, and sickle-cell anemia. In these embodiments, the RBCs can include UCB cells, e.g., autologous UCB cells.

Unless otherwise defined, all technical terms used herein have the same meaning as commonly understood by one of ordinary skill in the art to which this invention belongs.

The phrases “isolated” or “biologically pure” refer to material which is substantially or essentially free from components which normally accompany it as found in its native state.

The terms “agent” and “therapeutic agent” as used herein refer to a chemical entity or biological product, or combination of chemical entities or biological products, administered to a subject (a mammal such as a human) to treat a disease or condition (e.g., hypoxia, ischemia, anemia, etc.). Examples of therapeutic agents include packed RBC blood products.

By the term “freeze-resistant sterile container” is meant any sterile container that is resistant to freezing and suitable for containing cells.

As used herein, “packed blood cells” and “packed RBCs” refer to a concentrated preparation of red blood cells that is obtained from whole blood (e.g., UCB) by removing the plasma and mononuclear cells (as by centrifugation).

The terms “patient,” “subject” and “individual” are used interchangeably herein, and mean a subject, typically a mammal, to be treated, diagnosed, and/or to obtain a biological sample from. Subjects include, but are not limited to, humans, non-human primates, horses, cows, sheep, pigs, rats, mice, insects, dogs, and cats. A human in need of treatment for anemia, ischemia, hypoxia, a hemoglobin disorder, or a hematopoietic disorder is an example of a subject.

By “therapeutically effective amount” is meant an amount of a composition or packed RBC blood product of the present invention effective to yield the desired therapeutic response without undue adverse side effects (such as toxicity, irritation, or allergic response). For example, an amount effective to reduce, alleviate or eliminate anemia, hypoxia, ischemia, a hematopoietic disorder, etc. in a subject. The specific therapeutically effective amount will vary with such factors as the particular condition being treated, the physical condition of the patient, the type of mammal or animal being treated, the duration of the treatment, and the nature of concurrent therapy (if any).

As used herein, the terms “therapeutic treatment” and “therapy” are defined as the application or administration of a therapeutic agent (e.g., a packed RBC blood product as described herein) or therapeutic agents to a patient who has a disease, a symptom of disease or a predisposition toward a disease, with the purpose to cure, heal, alleviate, relieve, alter, remedy, ameliorate, improve or affect the disease, the symptoms of disease, or the predisposition toward disease.

Although kits, compositions, and methods similar or equivalent to those described herein can be used in the practice or testing of the present invention, suitable kits, compositions, and methods are described below. All publications, patent applications, and patents mentioned herein are incorporated by reference in their entirety. In the case of conflict, the present specification, including definitions, will control. The particular embodiments discussed below are illustrative only and not intended to be limiting.

### DETAILED DESCRIPTION

Described herein are compositions and kits for low temperature storage of a specified quantity of packed RBCs that include L-carnitine, HES and blood plasma proteins. Methods of storing a packed RBC blood product, transfusing a packed RBC blood product into a subject, and treating anemia, ischemia, hypoxia, a hemoglobin disorder, or a hematopoietic disorder in a subject (e.g., a human), are also described herein. RBCs frozen in L-carnitine, HES, and blood plasma proteins such as HSA are more active metabolically, more durable, and longer-lived after transfusion than RBCs frozen in glycerol solutions. Due to the low toxicity of L-carnitine/HES/HSA, RBCs do not have to be washed before infusion. UCB RBCs have high concentrations of fetal hemoglobin that binds oxygen at lower tensions than adult hemoglobin and deliver more oxygen to the ischemic or hypoxic brain or other organs (heart, ischemic limb). The combination of L-carnitine, HES and HSA lowers individual concentrations and reduces toxicity of each individual chemical so they protect the cells before, during, and after freezing, allowing the RBCs to be thawed and transfused without washing. The use of packed RBCs reduces potassium depletion and improves the metabolic status of the cells so that they have a longer life after transfusion. Freeze-drying the L-carnitine, HES and HAS and placing them inside a sterile freezing container (e.g., bag) reduces the expense of the reagents and container.

### Kits and Compositions for Storage of Blood Cells

L-carnitine is a natural amino acid, an antioxidant, and zwitterion that enters cells to prevent ice formation. At 6% concentration, L-carnitine allows 80% recovery of RBCs but prolonged incubation and prolonged incubation in 3% L-carnitine is non-toxic (Zhai, et al. 2017). L-carnitine is neuroprotective (Virmani, et al. 2013) in stroke (Zhang, et al. 2012; Wang, et al., 2016) and neonatal hypoxia-ischemia (Ferreira, et al. 2017). Hydroxyethyl starch (HES, 130/0.4) is added to reduce the concentration of L-carnitine needed for cryoprotection. At 6% concentration, HES improves cell survival during freezing and thawing but also reduces oxidative stress and inflammatory response in hemorrhagic shock (Chen, et al. 2013), and is used to treat patients after heart surgery (Tobey, et al. 2017). Human serum albumin (HSA) is the most abundant protein in plasma, constituting as much as 50% of plasma proteins. Due to its molecular size, albumin remains in the extracellular space and draws water out of the cells, reducing cell edema.

Described herein are kits for storing a packed RBC blood product, and kits for transfusing a packed RBC blood product into a subject. Kits for low temperature storage of a specified quantity of packed RBCs include a sterile freeze-resistant container (e.g., a bag) that includes L-carnitine, HES, and blood plasma proteins. In a sterile container as described herein, the L-carnitine and HES are present in quantities effective to provide cryoprotection for the specified quantity of packed RBCs and the blood plasma proteins are present in a quantity effective to reduce RBC edema for the specified quantity of packed RBCs. In some embodiments, the blood plasma proteins are HSA. In some embodiments, the L-carnitine, HES and blood plasma proteins are freeze-dried. In some embodiments, the HES has a molecular weight between about 130 and about 200 (e.g., between about 130 and about 135) kDa and between about 35% and about 50% (e.g., between about 40% and about 45%) of the starch glucose units have been modified with hydroxyethyl groups. Any suitable container, such as a sterile bag, for example, can be used. Sterile containers can be made of any suitable material or materials, including plastic, rubber, silicone, and composite materials.

In the kits described herein, the sterile freezing container typically includes packed RBCs. In some embodiments, the packed RBCs are essentially free of white blood cells. The packed RBCs can include, for example, UCB cells. In a typical embodiment, packed RBCs are added to sterile bags containing freeze-dried L-carnitine, HES, and albumin. Serum has long been known to be cryoprotective, and albumin is the most common plasma protein. Albumin not only provides osmotic protection of cells but stimulates proliferation of cells by activation of the Jak/stat pathway. Together, these two natural substances preserve RBCs stored at room temperature, at 4° C., and at all other temperatures during freezing. The compositions, containers and kits described herein allow freezing of umbilical cord blood (UCBs) in a closed system. The closed system would be represented by a bag with at least three tubes, the first would allow input of the RBC's, a second would allow wash and other solutions, and a third allows the RBC's to come out. The first can be sealed at multiple points to trap blood between the seals for ABO and other tests of the RBC. The bag would contain identification of the bag and its contents.

A kit as described herein may also include materials for thawing, washing and reconstituting the blood product. Kits also typically include packaging and instructional materials for preparation and use of the kit components. While the instructional materials typically include written or printed materials, they are not limited to such. Any medium capable of storing such instructions and communicating them to an end user is encompassed by the kits herein.

In the kits described herein, the L-carnitine, HES and blood plasma proteins can be combined in a composition within the freeze-resistant sterile container. In other embodiments, they are individually added to the freeze-resistant sterile container. In some embodiments of a composition as described herein, the L-carnitine, HES and blood plasma proteins are freeze-dried.

Also described herein are compositions for storing a packed RBC blood product, and compositions for transfusing a packed RBC blood product into a subject. In a composition as described herein, any suitable blood plasma proteins can be used. In a typical embodiment, HSA is used as the blood plasma proteins. In a typical embodiment, HES 130/0.4 is used. However, any suitable HES can be used. HES is a general term and can be sub-classified according to average molecular weight, molar substitution, concentration, C2/C6 ratio and Maximum Daily Dose. In the example of HES 130/0.4, the ‘130’ indicates the molecular weight, and the ‘0.4’ indicates the molar substitution. In some embodiments, the HES has a molecular weight between about 130 and about 200 (e.g., between about 130 to about 135) kDa and between about 35% and about 50% (e.g., between about 40% to about 45%) of the starch glucose units have been modified with hydroxyethyl groups.

In one embodiment, because HSA protects RBCs at a 2% concentration for example, a composition for storage of blood cells includes 2% L-carnitine, 2% HES, and 2% albumin for minimizing toxicity while maximizing protection of RBCs during freezing.

### Methods of Storing a Packed RBC Blood Product

In embodiments, methods of storing a packed RBC blood product include the steps of: mixing the blood product with L-carnitine, HES and blood plasma proteins in a sterile freeze-resistant container as described herein, incubating the blood product preparation in the sterile container for a period of time effective to provide cryoprotection to the blood product and reduce RBC edema (e.g., approximately two weeks); and freezing the container and the blood product at a temperature between about −20° C. and about −180° C. In some embodiments, the step of freezing the blood product includes a controlled rate of freezing of about 1° C. per minute. In the methods, the RBCs can include UCB cells, and the blood plasma proteins can be HSA. In a typical embodiment, the L-carnitine, HES and blood plasma proteins are freeze-dried. Generally, the HES has a molecular weight between about 130 and about 200 (e.g., between about 130 and about 135) kDa and between about 35% and about 50% (e.g., between about 40% and about 45%) of the starch glucose units have been modified with hydroxyethyl groups.

The methods of storing a packed RBC blood product involve a combination of HES and L-carnitine to replace glycerol as a cryopreservant for UCB cells. Generally in the methods, UCB that is hemolyzed is not frozen but is freeze-dried for fetal hemoglobin (HbF). RBCs combined with HES and L-carnitine can be directly infused intravenously without needing to be washed. Frozen RBCs have long been a Food and Drug Administration (FDA)-licensed product. Washed RBCs are particularly useful in patients with Immunoglobulin A (IgA) deficiencies and patients who have allergic reactions to blood products. UCB RBCs are particularly useful for treating neonatal anemia which affects 6% of infants. Such infants can be infants suffering from Rh disease, also known as hemolytic disease of the newborn (HDN). In some countries, such as India, about 15% of the mothers are Rh−. A child born of a Rh− mother and an Rh+ father may be severely anemic in utero or shortly after birth. The preferred treatment of such a child is to infuse Rh− umbilical cord blood, which contains fetal hemoglobin that capture oxygen at lower partial pressures.

In some alternative embodiments, the compositions, containers and kits are used in methods to increase longevity of non-frozen RBCs. The longevity of non-frozen RCSs is extended by adding adenosine and L-carnitine to the sterile container.

### Methods of Transfusing a Packed RBC Blood Product Into a Subject

A method of transfusing a packed RBC blood product into a subject (e.g., a human) includes thawing, washing and reconstituting a blood product stored according to the methods described herein, and infusing the reconstituted blood product into the subject. In the method, the blood product is ABO-matched to the subject. In a method of transfusing, the RBCs can be UCB cells, the blood plasma proteins can be HSA, and the L-carnitine, HES and blood plasma proteins can be freeze-dried. Generally, the HES has a molecular weight between about 130 and about 200 (e.g., between about 130 and about 135) kDa and between about 35% and about 50% (e.g., between about 40% and about 45%) of the starch glucose units have been modified with hydroxyethyl groups. Any suitable transfusion protocol can be used, and transfusion protocols are well known in the art. Methods of transfusing blood and blood products into humans are well known, and described, for example, in Carson et al., Annals of Internal Medicine. 157 (1): 49-58, 2012; D. Cherkas, Emergency Medicine Practice. 13 (11), 2011; and Sharma et al., Am Fam Physician. 83(6):719-724, 2011. The packed RBC blood products as described herein can be directly transfused intravenously without requiring washing. The subject receiving the transfusion is typically a human (e.g., neonate, infant, adult human) suffering from anemia, hypoxia, ischemia, a hematopoietic disorder, etc. In a typical embodiment in which the packed RBC blood product contains few or no white blood cells (e.g., free of white blood cells), the likelihood of activating an undesirable immune response is minimal. A method of transfusing a packed RBC blood product into a subject may include a single transfusion or multiple transfusions.

### Methods of Treating Anemia, Ischemia, Hypoxia, and Hemoglobin and Hematopoietic Disorders In a Subject

A method of treating anemia, ischemia, hypoxia, a hemoglobin disorder, or a hematopoietic disorder in a subject (e.g. human) includes thawing, washing and reconstituting a packed RBC blood product stored according to the storage methods described herein, and infusing the reconstituted blood product into the subject. In the method, the blood product is ABO-matched to said subject. In some embodiments, the blood product includes cells from the subject. In embodiments of a method of treatment, the RBCs can be UCB cells, the blood plasma proteins can be HSA, and the L-carnitine, HES and blood plasma proteins can be freeze-dried. Generally, the HES has a molecular weight between about 130 and about 200 (e.g., between about 130 and about 135) kDa and between about 35% and about 50% (e.g., between about 40% and about 45%) of the starch glucose units have been modified with hydroxyethyl groups. In a typical embodiment, the subject is suffering from at least one of the following disorders: ischemia, hypoxia, anemia, thalassemia, and sickle-cell anemia. In some embodiments, the subject is a human infant suffering from the disorder(s), and in these embodiments, the RBCs can include UCB cells. The UCB cells may be autologous UCB cells. In other embodiments, the subject is an adult human suffering from the disorder(s). Any suitable transfusion protocol can be used. A method of treating anemia, ischemia, hypoxia, a hemoglobin disorder, or a hematopoietic disorder in a subject may include a single transfusion or multiple transfusions.

The packed RBC blood products stored according to the methods described herein can be used to treat any type of disorder associated with low blood cell levels or abnormal blood cells. UCB RBCs stored and frozen by the storage methods described herein can be used to treat, for example, ischemic infants (e.g., ischemic human infants). UCB cells are particularly suitable for treating infants, particularly infants that have anemia, hypoxia, or ischemia, as well as infants with hematopoietic disorders such as thalassemia and sickle cell anemia. Regarding adult RBCs, most blood banks discard the red cell fraction (RCF) of UCB and often do not freeze their cord blood with common ABO types and try to use them for up to 2 weeks after collection. They freeze cord blood with rare ABO, such as 0-negative, using the traditional glycerolization for cryopreservation. In contrast, the compositions, containers and methods described herein increase the convenience and reduce the cost of cryopreserving RBCs. By providing a convenient and inexpensive approach to freezing cord blood and adult blood, all the UCB RBCs can be cryopreserved.

The methods described herein can further include detecting a state or condition of one or more of ischemia, hypoxia, anemia, thalassemia, and sickle-cell anemia in the subject. The detection is typically done prior to transfusing a packed RBC blood product as described herein into the subject. Methods of detecting ischemia, hypoxia, anemia, thalassemia, and sickle-cell anemia in a subject are well known in the art. The methods can further include monitoring treatment progress. In such an embodiment, the method includes the step of determining a level of change in one or more suitable parameters or markers depending upon the disease or disorder being treated, using, for example, one or more diagnostic markers or diagnostic measurement (e.g., screen, assay) in a subject suffering from or susceptible to a disorder or symptoms thereof associated with low blood cell levels or abnormal blood cells who has received a transfusion of a therapeutic amount of a packed RBC blood product as described herein. The level of marker determined in the method can be compared to known levels of marker in either healthy normal controls or in other afflicted patients to establish the subject's disease status. In preferred embodiments, a second level of marker in the subject is determined at a time point later than the determination of the first level, and the two levels are compared to monitor the course of disease or the efficacy of the therapy.

### EXAMPLES

The present invention is further illustrated by the following specific examples. The examples are provided for illustration only and should not be construed as limiting the scope of the invention in any way.

### Example 1

### Method of Storing Packed RBCs

Packed RBCs are placed inside a sterile freezing bag (a sterile freeze-resistant container) containing appropriate amounts of freeze-dried L-carnitine, HES, and albumin that result in 2-3% concentrates of each of the three cryopreservants when a specified volume of packed RBCs is placed in the bag. The cells are gently shaken to mix the cryopreservants, allowed to incubate for an hour, and frozen with a controlled rate freezer at 1° C./minute to −20° C. or −180° C., depending on the planned storage time. When needed, the cells are thawed at room temperature and directly administered to patients.

### Example 2

### Methods of Treatment

The RBC's are ABO-matched with the recipient, thawed, and infused directly into the recipient. A particular therapeutic target is neonatal anemia (which affects almost all infants born more than 4 weeks prematurely), post-natal hypoxia and ischemia, and hematopoietic disorders. Autologous use of these cells is particularly suitable.

The RBCs can be used as a blood transfusion to treat anemia. Because blood contains HbF, they bind oxygen at lower levels than adult hemoglobin (HbA) and are useful for patients with anoxia or ischemia, including stroke and critical limb ischemia. Finally, UCB would be particularly useful for treating infants with hemoglobin disorders such as thalassemia and sickle-cell anemia. Because the preparation eliminates or minimizes white blood cells, the risk of activation of immune responses in the patient should be minimal.

### Example 3

### Additional Uses of Stored Packed RBCs

Researchers will be able to study UCB and other RBCs frozen in L-carnitine, HES, and HSA. There is currently no source of UCB RBCs for research. For example, if researchers are interested in the behavior and competition of UCB cells collected from births of infants with normal, thalassemic, sickle cell, or other abnormal RBCs, such cells are not available. Studies of UCB RBCs should provide information concerning how RBCs deliver oxygen to the infants in utero.

### Other Embodiments

Any improvement may be made in part or all of the kits, containers, compositions, and methods. All references, including publications, patent applications, and patents, cited herein are hereby incorporated by reference. The use of any and all examples, or exemplary language (e.g., “such as”) provided herein, is intended to illuminate the invention and does not pose a limitation on the scope of the invention unless otherwise claimed. Any statement herein as to the nature or benefits of the invention or of the preferred embodiments is not intended to be limiting, and the appended claims should not be deemed to be limited by such statements. More generally, no language in the specification should be construed as indicating any non-claimed element as being essential to the practice of the invention. This invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contra-indicated by context.

1. A kit for low temperature storage of a specified quantity of packed red blood cells (RBCs), the kit comprising:
i) a freeze-resistant sterile container, the sterile container comprising L-carnitine, hydroxyethyl starch (HES), and blood plasma proteins, wherein the L-carnitine and HES are present in quantities effective to provide cryoprotection for the specified quantity of packed RBCs and the blood plasma proteins are present in a quantity effective to reduce RBC edema for the specified quantity of packed RBCs;
ii) packaging; and
iii) instructions for use.

2. The kit of claim 1, wherein the blood plasma proteins consist essentially of Human Serum Albumin (HSA), and the L-carnitine, HES and blood plasma proteins are freeze-dried.

3. (canceled)

4. The kit of claim 1, further comprising packed RBCs.

5. The kit of claim 1, wherein the HES has a molecular weight between about 130 and about 200 kDa and between about 35% and about 50% of the starch glucose units have been modified with hydroxyethyl groups.

6. (canceled)

7. The kit of claim 4, wherein the packed RBCs comprise umbilical cord blood (UCB) RBCs, and are essentially free of white blood cells.

8. (canceled)

9. A method of storing a packed RBC blood product, the method comprising the steps of:
i. mixing the blood product with L-carnitine, HES and blood plasma proteins in the freeze-resistant sterile container of claim 1;
ii. incubating the blood product preparation in the sterile container for a period of time effective to provide cryoprotection to the blood product and reduce RBC edema; and
iii. freezing the sterile container and the blood product at a temperature between about −20° C. and about −180° C.

10. The method of claim 9, wherein step iii. of freezing the blood product comprises a controlled rate of freezing of about 1° C. per minute.

11. The method of claim 9, wherein the RBCs comprise UCB cells.

12. The method of claim 9, wherein the blood plasma proteins consist essentially of HAS, and the L-carnitine, HES and blood plasma proteins are freeze-dried.

13. (canceled)

14. The method of claim 9, wherein the HES has a molecular weight between about 130 and about 200 kDa and between about 35% and about 50% of the starch glucose units have been modified with hydroxyethyl groups.

15. A method of transfusing a packed RBC blood product into a subject comprising thawing, washing and reconstituting a blood product stored according to the method of claim 9, and infusing the reconstituted blood product into the subject, wherein said blood product is ABO-matched to said subject.

16. The method of claim 15, wherein the RBCs comprise UCB cells.

17. The method of claim 15, wherein the blood plasma proteins consist essentially of HAS, and the L-carnitine, HES and blood plasma proteins are freeze-dried.

18. (canceled)

19. The method of claim 15, wherein the HES has a molecular weight between about 130 and about 200 kDa and between about 35% and about 50% of the starch glucose units have been modified with hydroxyethyl groups.

20. A method of treating anemia, ischemia, hypoxia, a hemoglobin disorder, or a hemato-poietic disorder in a subject, the method comprising thawing, washing and reconstituting a packed RBC blood product stored according to the method of claim 9, and infusing the reconstituted blood product into the subject, wherein said blood product is ABO-matched to said subject.

21. The method of claim 20, wherein the blood product comprises cells from the subject, and the RBCs comprise UCB cells.

22. (canceled)

23. The method of claim 20, wherein the blood plasma proteins consist essentially of HAS, and the L-carnitine, HES and blood plasma proteins are freeze-dried.

24. (canceled)

25. The method of claim 20, wherein the HES has a molecular weight between about 130 and about 200 kDa and between about 35% and about 50% of the starch glucose units have been modified with hydroxyethyl groups.

26. The method of claim 20, wherein the subject is suffering from at least one disorder selected from the group consisting of: ischemia, hypoxia, anemia, thalassemia, and sickle-cell anemia.

27. The method of claim 20, wherein the subject is a human infant suffering from at least one disorder selected from the group consisting of: ischemia, hypoxia, anemia, thalassemia, and sickle-cell anemia.

28. The method of claim 27, wherein the RBCs comprise autologous UCB cells.

29. (canceled)
